(19) United States
(12) Patent Application Publication
(10) Pub. No.: US 2023/0346486 A1
(43) Pub. Date: Nov. 2, 2023

(54) MEDICAL OPERATION ASSISTANCE SYSTEM

(71) Applicant: TERUMO KABUSHIKI KAISHA, Tokyo (JP)

(72) Inventor: Yuichi HIOKI, Fuji Shizuoka (JP)

(21) Appl. No.: 18/346,848

(22) Filed: Jul. 4, 2023

Related U.S. Application Data
(63) Continuation of application No. PCT/JP2022/001482, filed on Jan. 18, 2022.

(30) Foreign Application Priority Data
Jan. 20, 2021 (JP) 2021-007380

(51) Int. Cl. A61B 34/20; G06T 7/00

(52) U.S. Cl. CPC A61B 34/20; G06T 7/0012; G06T 2207/10132; G06T 2207/20081; G06T 2207/30101; A61B 2090/3788

(57) ABSTRACT

A system for assisting in performing a medical operation on an organ, includes a display, a ultrasonic probe, and a processor configured to: upon receipt of echo signals from the probe, which are generated in response to ultrasound signals transmitted by the probe toward the organ, generate a first cross-sectional image along a first direction and a second cross-sectional image along a second direction, determine candidates of a first puncture line in the first image, input the first image and each candidate to a first learning model and acquire first evaluation information for each candidate, determine candidates of a second puncture line in the second image, input the second image and each candidate to a second learning model and acquire second evaluation information for each candidate, and control the display to display a first screen showing each candidate associated with the corresponding image and evaluation information.

### CROSS-REFERENCE TO RELATED APPLICATION

This application is a continuation of International Patent Application No. PCT/JP2022/001482 filed Jan. 18, 2022, the entire contents of which are incorporated herein by reference.

### FIELD

Embodiments described herein relate generally to a medical operation assistance system for assisting in performing a medical operation involving a puncture device, and a non-transitory computer readable medium storing a program causing a computer to execute a method of assisting in performing such a medical operation.

### BACKGROUND

In the related art, an ultrasonic image generation apparatus is widely used for medical diagnoses and examinations. The ultrasonic image generation apparatus has an ultrasonic probe, and emits ultrasound signals from the ultrasonic probe to a subject and generates a tomographic image of the subject based on the echo signals received through the ultrasonic probe.

Further, a puncture technique is widely performed in which a physician punctures a desired site using a puncture device such as a puncture needle while observing the tomographic image of the subject, and a technique for assisting such a puncture technique has been developed. For example, there is a puncture assistance system that provides, when an ultrasonic image of a blood vessel to be punctured is acquired, information on a collapsed state of the blood vessel due to pressing of an ultrasonic probe so that a physician or a robot can puncture the blood vessel quickly and accurately.

### SUMMARY OF THE INVENTION

However, such a puncture assistance system merely determines the ease of puncture according to the current pressing state of the ultrasonic probe, and insertion of the puncture device into the subject by the operator is not suitably assisted.

Embodiments of the disclosure provide a medical operation assistance system and the like capable of suitably assisting the puncture operation.

In one embodiment, a medical operation assistance system for assisting in performing a medical operation on an organ, comprises: a display device; an ultrasonic probe; and one or more processors. The processors are configured to, upon receipt of echo signals from the ultrasonic probe, which are generated in response to ultrasound signals transmitted by the ultrasonic probe toward the organ, generate a first image of a first cross section of the organ along a first direction and a second image of a second cross section of the organ along a second direction orthogonal to the first direction, determine a plurality of candidates of a first puncture line indicating an inserting position and inserting direction of the puncture device in the first image, input the first image and information indicating each of the candidates of the first puncture line to a first machine learning model, and acquire first evaluation information output by the first machine learning model for each of the candidates of the first puncture line, determine a plurality of candidates of a second puncture line indicating an inserting position and inserting direction of the puncture device in the second image, input the second image and information indicating each of the candidates of the second puncture line to a second machine learning model, and acquire second evaluation information output by the second machine learning model for each of the candidates of the second puncture line, and control the display device to display a first screen in which each of the candidates of the first and second puncture lines is displayed in association with the corresponding image and evaluation information.

According to the disclosure, it is possible to suitably assist an operator who employs a puncture device during a puncture operation.

### BRIEF DESCRIPTION OF THE DRAWINGS

FIG. 1 is a schematic diagram of an assistance system according to a first embodiment.

FIG. 2 is a block diagram illustrating a configuration example of the assistance system.

FIG. 3 is a diagram illustrating outlines of a first learning model and a second learning model.

FIG. 4 is a diagram illustrating an example of information stored in a training data database (DB).

FIG. 5 is a flowchart illustrating an example of a training data generating procedure.

FIG. 6 is a schematic diagram illustrating an example of a reception screen.

FIG. 7 is a flowchart illustrating an example of a learning model generating procedure.

FIG. 8 is a flowchart illustrating an example of an assistance information outputting procedure.

FIG. 9 is a schematic diagram illustrating an example of a screen displayed on a display device.

FIG. 10 is a schematic diagram illustrating an example of the screen displayed on the display device.

FIG. 11 is a schematic diagram illustrating configurations of a first learning model and a second learning model according to a second embodiment.

FIG. 12 is a diagram illustrating a flow of a process according to a third embodiment.

FIG. 13 is a flowchart illustrating an example of a training data generating procedure according to the third embodiment.

FIG. 14 is a flowchart illustrating an example of an assistance information outputting procedure according to the third embodiment.

FIG. 15 is a flowchart illustrating an example of a retraining process of learning models according to a fourth embodiment.

### DETAILED DESCRIPTION

Embodiments of the disclosure will be specifically described with reference to the drawings.

### First Embodiment

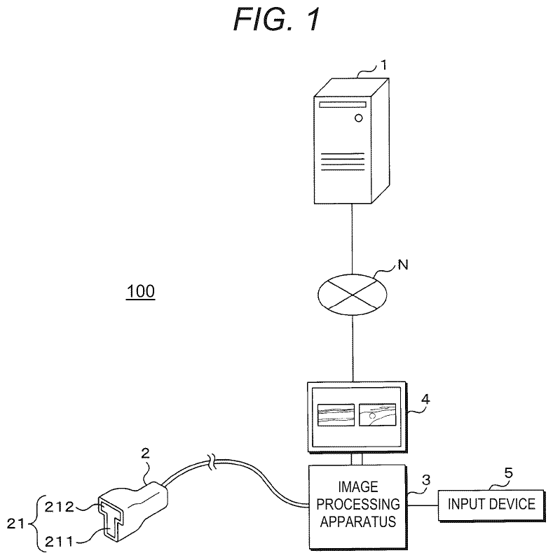

FIG. 1 is a schematic diagram of a medical operation assistance system 100 according to a first embodiment. The assistance system 100 includes an information processing apparatus 1, an ultrasonic probe 2, and an image processing apparatus 3. The ultrasonic probe 2 and the image processing apparatus 3 are connected to each other in a wired or wireless manner, and can exchange various signals. The information processing apparatus 1 and the image processing apparatus 3 are communicatively connected to a network N such as a local area network (LAN) and/or the Internet.

The assistance system 100 according to the present embodiment generates, based on an ultrasonic image of a subject to be punctured, assistance information for assisting puncture into the subject. For example, an operator such as a physician inserts a puncture device such as a puncture needle into a target site and punctures a blood vessel using the generated assistance information.

The ultrasonic probe 2 is a scanning device that scans an organ of the subject with ultrasound, and ultrasound scanning is controlled by the image processing apparatus 3. The ultrasonic probe 2 includes, for example, a transducer array 21 including a plurality of transducer elements, and an acoustic matching layer and an acoustic lens that are not illustrated. The transducer array 21 transmits ultrasound signals in response to a driving signal output from the image processing apparatus 3. The ultrasound signals are transmitted from the ultrasonic probe 2 to the living body of the subject via the acoustic matching layer and the acoustic lens. The acoustic matching layer is a member for matching acoustic impedance between the transducer array 21 and the subject. The acoustic lens is an element for converging the ultrasounds spreading from the transducer array 21 and transmitting the ultrasounds to the subject. The ultrasounds transmitted from the ultrasonic probe 2 to the subject are reflected by discontinuous surfaces of the acoustic impedance in the organ of the subject, and are received by the transducer array 21. An amplitude of the reflected waves depends on a difference in the acoustic impedance on a reflective surface. An arrival time of the reflected waves depends on a depth of the reflective surface. The transducer array 21 converts a vibration pressure of the reflected ultrasounds into an electric signal. Hereinafter, the electric signal will be referred to as an echo signal. The ultrasonic probe 2 outputs the echo signal to the image processing apparatus 3.

The image processing apparatus 3 generates an ultrasound tomographic image based on the echo signal from the ultrasonic probe 2. In the present embodiment, the ultrasonic probe 2 is brought into contact with the skin of the subject to be punctured, and an ultrasound tomographic image of an in-vivo portion including a blood vessel under the skin in contact with the ultrasonic probe 2 is generated. The image processing apparatus 3 includes a display device 4 for displaying the generated ultrasound tomographic image and the assistance information corresponding to the ultrasound tomographic image to the physician or the like, and an input device 5 for receiving an input operation from the physician or the like.

The ultrasonic probe 2 according to the present embodiment is a T-shaped ultrasonic probe that includes the transducer array 21 including a first transducer array 211 and a second transducer array 212. The first transducer array 211 and the second transducer array 212 are arranged orthogonal to each other in the same plane on the substantially flat bottom surface of the main body of the ultrasonic probe 2. The first transducer array 211 and the second transducer array 212 respectively scan the organ of the subject corresponding to the arrangement directions. The image processing apparatus 3 generates, based on the echo signals received through the first transducer array 211 and the second transducer array 212 at the same time, a first image (i.e., a first ultrasound tomographic image) illustrating a cross section of the in-vivo portion of the subject in the first direction and a second image (i.e., a second ultrasound tomographic image) illustrating a cross section in the second direction orthogonal to the first direction. For example, the ultrasonic probe 2 is brought into contact with a surface of the skin of the subject such that a lower end portion of the T shape is directed toward a (proximal end of the blood vessel, an upper end portion of the T shape is directed toward a distal end of the blood vessel, and the first transducer array 211 is moved along a running direction of the blood vessel. Accordingly, the image processing apparatus 3 can generate the first image (i.e., a long-axis cross-sectional image) that is a cross-sectional image in the first direction along a long axis direction (i.e., the running direction) of the blood vessel, and the second image (i.e., a short-axis cross-sectional image) that is a cross-sectional image in the second direction indicating a direction orthogonal to the first direction, that is, a short axis direction of the blood vessel, at the same time point. For example, a blood vessel, a subcutaneous tissue, a skin surface, a thrombus, and a calcified lesion are included in the first image and the second image.

The ultrasonic probe 2 is not limited to the T-shaped ultrasonic probe described above. When a one-axis ultrasonic probe is used, the first image and the second image may be acquired by acquiring an echo signal in the first direction along the long axis direction of the blood vessel in the subject, and then continuously acquiring an echo signal in the second direction orthogonal to the first direction.

The information processing apparatus 1 performs various types of information processes and performing information transmission and reception, and is, for example, a server computer, or a personal computer. The information processing apparatus 1 may be a local server installed in a facility (e.g., a hospital or the like) at which the image processing apparatus 3 and the ultrasonic probe 2 are used, or may be a cloud server that is communicatively connected via the Internet or the like. The information processing apparatus 1 generates the assistance information based on the ultrasound tomographic image acquired via the image processing apparatus 3. The information processing apparatus 1 outputs the generated assistance information to the image processing apparatus 3 and causes the display device 4 to display the assistance information.

The assistance information is information for assisting the puncture into the subject, and includes, for example, information on a puncture line indicating an inserting position and an inserting direction of a puncture device such as a puncture needle to be punctured into the subject. More specifically, the assistance information includes information on the puncture line indicating the inserting position and the inserting direction or evaluation information such as an evaluation score for the puncture line. The information processing apparatus 1 generates the assistance information corresponding to the first image and the second image by using the learning models to be described later. That is, the information processing apparatus 1 generates information on the puncture line suitable for a situation of the blood vessel or the like of the subject indicated in the first image and the second image. For example, an ultrasound impermeable marker is mounted to the distal end of the puncture device, and a position of the puncture device is visualized in the first image and the second image. The operator such as a physician can suitably puncture the blood vessel while comparing the information on the puncture lines displayed on the first image and the second image with the position of the puncture device.

The assistance system 100 according to the present embodiment is particularly suitably applied to puncture into a lower limb blood vessel (for example, a superficial femoral artery) having a long distance from the skin surface and a large blood flow rate. By using the assistance information provided by the assistance system 100, even an unskilled operator can perform the same puncture content as that performed by a skilled operator in a case where a high skill level is required for specifying a puncture line such as in lower limb puncture.

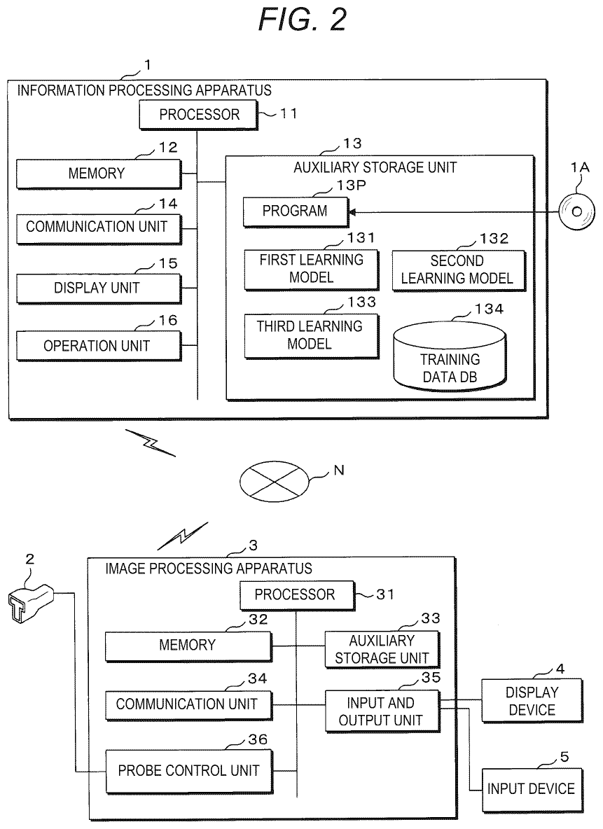

FIG. 2 is a block diagram illustrating a configuration example of the assistance system 100. The information processing apparatus 1 includes a processor 11, a main memory 12, an auxiliary storage unit 13, a communication unit 14, a display unit 15, and an operation unit 16. The information processing apparatus 1 may include a plurality of computers, or may be a virtual machine virtually operated by software.

The processor 11 includes one or more arithmetic processing circuits such as central processing units (CPUs) or graphics processing units (GPUs). The processor 11 reads and executes a program 13P stored in the auxiliary storage unit 13, thereby causing the information processing apparatus 1 to perform various processes related to the generation of the assistance information.

The main memory 12 is a static random access memory (SRAM), a dynamic random access memory (DRAM), or a flash memory. The main memory 12 temporarily stores the program 13P loaded from the auxiliary storage unit 13 when an arithmetic process of the processor 11 is executed by the processor 11, and various data generated by the arithmetic process of the processor 11.

The auxiliary storage unit 13 is a nonvolatile storage device, such as a hard disk, an electrically erasable programmable ROM (EEPROM), or a flash memory. The auxiliary storage unit 13 may be an external storage device connected to the information processing apparatus 1. The auxiliary storage unit 13 stores programs including the program 13P and data that are necessary for the processor 11 to execute various processes. In addition, the auxiliary storage unit 13 stores a first learning model 131, a second learning model 132, and a training data database (DB) 134. Each of the first learning model 131 and the second learning model 132 is a machine learning model that has been trained using training data. The first learning model 131 and the second learning model 132 are program modules constituting artificial intelligence software. Details of the learning models and the training data DB 134 will be described later. The auxiliary storage unit 13 may further store a third learning model 133. The third learning model 133 will be described in detail in another embodiment.

The program 13P may be recorded in a non-transitory computer readable storage medium 1A. The auxiliary storage unit 13 stores the program 13P copied from the storage medium 1A by a reader (not illustrated). The storage medium 1A is a semiconductor memory such as a flash memory, an optical disk, a magnetic disk, a magneto-optical disk, or the like. In addition, the program 13P according to the present embodiment may be downloaded from an external server (not illustrated) connected to a communication network and stored in the auxiliary storage unit 13.

The communication unit 14 is a communication interface (I/F) module for performing a process related to communication. The processor 11 controls the communication unit 14 to transmit and receive information to and from the image processing apparatus 3.

The display unit 15 is a display device that displays information such as the first image, the second image, and the assistance information described above. The display unit 15 is, for example, a liquid crystal display (LCD) or an organic electroluminescence (EL) display.

The operation unit 16 is an input device that receives an operation from a user. The input device is, for example, a keyboard or a pointing device such as a touch panel.

The image processing apparatus 3 includes a processor 31, a main memory 32, an auxiliary storage unit 33, a communication unit 34, an input and output unit 35, and a probe control unit 36.

The processor 31 includes one or more arithmetic processing circuits such as CPUs or GPUs. The main memory 32 is an SRAM, a DRAM, or a flash memory. The processor 31 performs various types of information processes by load and executing a program stored in the auxiliary storage unit 33.

The main memory 32 temporarily stores the program loaded from the auxiliary storage unit 33 when an arithmetic process is executed by the processor 31, and various data generated by the arithmetic process of the processor 31.

The auxiliary storage unit 33 is a nonvolatile storage device such as a hard disk, an EEPROM, or a flash memory. The auxiliary storage unit 33 stores programs and data that are necessary for the processor 31 to execute various processes. The auxiliary storage unit 33 may store the learning models described above.

The communication unit 34 is a communication interface module for performing a process related to communication. The processor 31 controls the communication unit 34 to transmit and receive information to and from the information processing apparatus 1 via the communication unit 34, and acquires the assistance information.

The input and output unit 35 is an input and output I/F circuit for connecting an external device. The display device 4 and the input device 5 are connected to the input and output unit 35. The display device 4 is, for example, an LCD or an organic EL display. The input device 5 is, for example, a keyboard or a pointing device such as a touch panel. The processor 31 controls the display device 4 to output the first image, the second image, the assistance information, and the like via the input and output unit 35. In addition, the processor 31 controls the input and output unit 35 to receive information input to the input device 5.

The probe control unit 36 includes a driving control unit, a transmission and reception control unit, an image generating unit, and the like that are not illustrated. The ultrasonic probe 2 is connected to the probe control unit 36. The probe control unit 36 controls an ultrasound scanning process performed by the ultrasonic probe 2. Specifically, the probe control unit 36 outputs a driving signal to the ultrasonic probe 2 to cause the ultrasonic probe 2 to generate the ultrasound signals and receive the echo signals. Further, the probe control unit 36 executes a process of generating the first image and the second image (i.e., the ultrasound tomographic images) based on the received echo signals. Each time the probe control unit 36 receives the echo signals, the probe control unit 36 generates a series of the first images and the second images in real time. The first image and the second image are, for example, B mode images in which an intensity of the reflected waves is represented by luminance, and two-dimensional tomographic images of the organ are reproduced. The types of the first image and the second image are not particularly limited. Since functions and a configuration of the probe control unit 36 are the same as those of an image processing apparatus commonly used in the related art, a detailed description thereof will be omitted. Further, the processor 31 may execute one or more of the functions of the probe control unit 36.

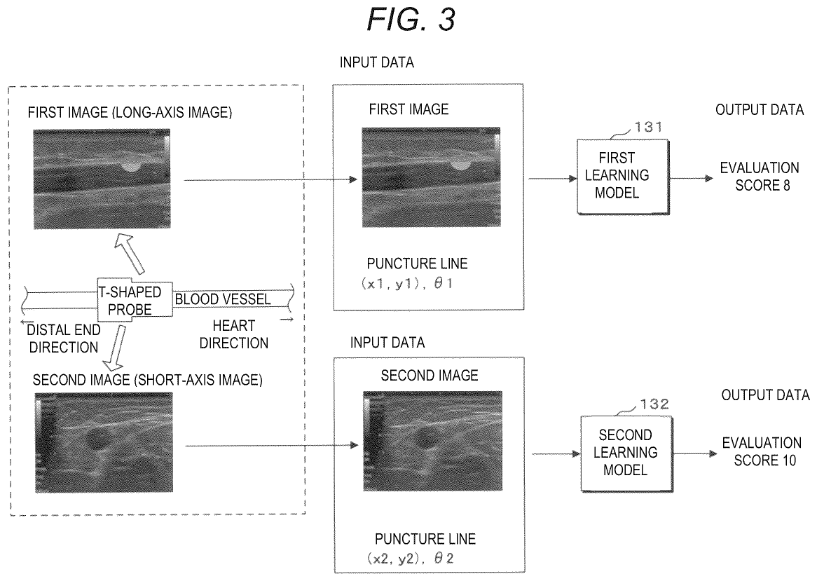

FIG. 3 is a diagram illustrating outlines of the first learning model 131 and the second learning model 132. The first learning model 131 is a software module that receives the first image indicating the cross section in the long axis direction of the blood vessel of the subject and a puncture line in the long axis direction of the blood vessel of the subject, and outputs information indicating an evaluation score for the puncture line. The second learning model 132 is a software module that receives the second image indicating the cross section in the short axis direction of the blood vessel of the subject and a puncture line in the short axis direction of the blood vessel of the subject, and outputs information indicating an evaluation score for the puncture line. Since the first learning model 131 and the second learning model 132 have the same configuration, the configuration of the first learning model 131 will be described below.

The information processing apparatus 1 performs machine learning operations for learning predetermined training data to generate the first learning model 131 in advance. Then, the information processing apparatus 1 inputs the first image of the subject acquired from the image processing apparatus 3 and the puncture line into the first learning model 131, and outputs the evaluation score for the puncture line.

For example, the first learning model 131 is a neural network model generated by deep learning, and is a convolutional neural network (CNN) that extracts a feature of an input image using many convolution layers. The first learning model 131 includes, for example, an input layer to which the first image and the puncture line are input, an intermediate layer that extracts a feature of an image, and an output layer that outputs information indicating an evaluation score.

The input layer of the first learning model 131 includes a plurality of nodes that receive an input of the first image and the puncture line included in an image portion, and transmits the input data to the intermediate layer. The intermediate layer includes a plurality of nodes that extracts features of the first image and the puncture line, and transmits the features extracted using various parameters to the output layer. The intermediate layer may include a convolution layer, a pooling layer, a fully coupled layer, and the like. The output layer includes one or more nodes that output the information indicating the evaluation score.

The input data input to the input layer of the first learning model 131 includes the first image and the puncture line. The puncture line is information indicating the puncture line for the first image. For example, the puncture line is defined by a coordinate value indicating one point (for example, a start point) on the puncture line and an angle indicating the inserting direction. The puncture line may be vectorized and input to the input layer. Further, the puncture line may be image data indicating the puncture line generated based on the coordinate value and the angle.

The output data output from the output layer of the first learning model 131 is the evaluation score for the puncture line. The evaluation score is indicated on, for example, a scale of one to ten, and the higher the score, the higher the evaluation, that is, the evaluation score indicates a puncture line having a low puncture risk. An evaluation form for the puncture line is not limited. The evaluation for the puncture line may be expressed, for example, as a percentage, or may be expressed as an evaluation ranking of a plurality of puncture lines, or the like.

The second learning model 132 has the same configuration as that of the first learning model 131, and receives the second image indicating the cross section in the short axis direction of the blood vessel of the subject and the puncture line in the short axis direction of the blood vessel of the subject, and outputs information indicating an evaluation score for the puncture line.

In the present embodiment, the first learning model 131 and the second learning model 132 are CNNs, but the configurations of the first learning model 131 and the second learning model 132 are not limited to the CNNs. The first learning model 131 and the second learning model 132 may be, for example, a neural network other than CNN, a support vector machine (SVM), a Bayesian network, or a learning model based on other learning algorithms such as regression trees.

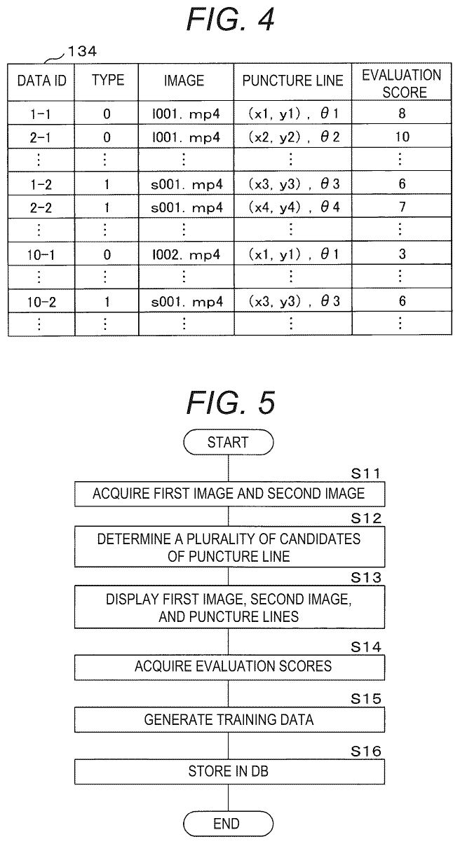

FIG. 4 is a diagram illustrating an example of information stored in the training data DB 134. The information processing apparatus 1 collects the training data for training the first learning model 131 and the second learning model 132, and stores the training data in the training data DB 134. The training data DB 134 stores, for example, a plurality sets of a data ID, a type, an image, a puncture line, and an evaluation score. The identification information for identifying each training data is stored in the data ID column. The information indicating the type of each training data is included in the type column. In the example of FIG. 4, the type column stores either identifier “0” indicating data in the long axis direction of the blood vessel of the subject or identifier “1” indicating data in the short axis direction of the blood vessel of the subject. The image column stores long-axis cross-sectional image data or short-axis cross-sectional image data of the blood vessel of the subject that is generated based on the echo signal of the ultrasonic probe 2. The coordinate values of the start point and the angle related to the puncture line are stored in the puncture line column. The length of the puncture line may further be stored in the puncture line column. The evaluation store for the puncture line is stored in the evaluation score column. Note that FIG. 4 is an example, and the structure of the training data DB 134 is not limited thereto.

In a learning phase that is a stage before an operation phase for performing puncture assistance, the information processing apparatus 1 generates the first learning model 131 and the second learning model 132 using the training data described above, and stores the generated first learning model 131 and second learning model 132. Further, in the operation phase, the information processing apparatus 1 uses the stored first learning model 131 and second learning model 132 to generate assistance information.

Hereinafter, processes performed by parts in the assistance system 100 having the configuration described above will be described. FIG. 5 is a flowchart illustrating an example of a training data generating procedure. The following process is executed in the learning phase by the processor 11 according to the program 13P stored in the auxiliary storage unit 13 of the information processing apparatus 1.

The processor 11 of the information processing apparatus 1 acquires the first image and the second image from the image processing apparatus 3 (step S11). The first image and the second image are ultrasonic tomographic images in the long axis direction and the short axis direction of the blood vessel, which have been generated based on the echo signals received by the ultrasonic probe 2 at the same time or at approximately the same time.

The processor 11 generates data indicating a plurality of candidates of the puncture line for each of the first image and the second image (step S12). For example, the processor 11 may select a predetermined number of puncture lines from a puncture line candidate table in which the start point and the angle of each puncture line are associated with each other according to a predetermined rule. Alternatively, the processor 11 may select a predetermined number of puncture lines from data in which a plurality of puncture lines have been registered by the physician or the like via the operation unit 16.

The processor 11 controls the display unit 15 to display a reception screen 151 that includes the acquired first image and second image, and the generated plurality of puncture lines (step S13). The processor 11 then acquires evaluation scores for the puncture lines (step S14).

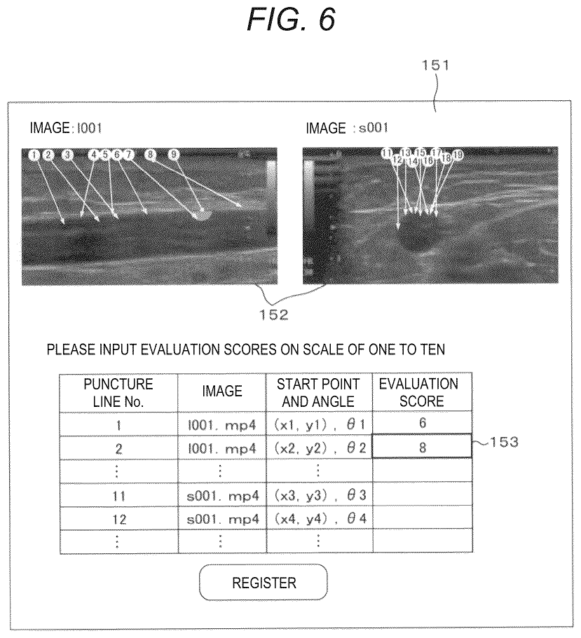

FIG. 6 is a schematic diagram illustrating an example of the reception screen 151. The reception screen 151 includes a puncture line display area 152, an evaluation score input area 153, a register button, and the like. The puncture line display area 152 displays the plurality of candidates of the puncture line on each of the first image and the second image in a superimposed manner. Each of the puncture lines is indicated by a drawing object such as a line based on the start point coordinates and the angle. The number for identifying each puncture line is also displayed together with the drawing object. In the example of FIG. 6, nine drawing objects each of which indicates a puncture line candidate and that have a different start point and angle are displayed on each of the first image and the second image. The evaluation score input area 153 displays a plurality of input boxes for receiving an input of the evaluation scores for the displayed puncture lines, in association with the corresponding numbers thereof. The physician or the like inputs the evaluation scores for the puncture lines. When the register button is tapped in a state where the evaluation scores for the puncture lines are input on the reception screen 151, the evaluation scores for the puncture lines are input to the operation unit 16. The processor 11 acquires the evaluation scores for the puncture lines.

The evaluation score for each puncture line is calculated according to a plurality of evaluation items. For example, in the long axis direction, the closer the angle of the puncture line with respect to the blood vessel is to 45 degrees, the higher the evaluation is. The less a thrombus or a calcified lesion exists on the puncture line, the higher the evaluation is. The less the meandering of the blood vessel in the periphery of the puncture line exists, the higher the evaluation is. In the short axis direction, the more the puncture line is perpendicular to the skin surface, the higher the evaluation is. The more the puncture line passes through a center of the blood vessel (i.e., a blood vessel puncture portion is at the center of the blood vessel), the higher the evaluation is. Further, the high evaluation indicates that the puncture risk is low. For example, the evaluation score according to these findings is calculated by a skilled physician or the like.

In the above description, the processor 11 is not limited to acquiring the evaluation scores for the puncture lines. The processor 11 may acquire an evaluation ranking of the puncture lines from the skilled physician or the like. In addition, the processor 11 may acquire selection of a predetermined number of puncture lines in association with the evaluation ranking in an evaluation descending order. Note that the processor 11 may automatically calculate the evaluation score based on the received evaluation ranking.

Description will be made again with reference to FIG. 5. The processor 11 generates training data that is a data set in which the evaluation score for each puncture line is labeled as a ground truth value for the first image or second image (step S15). The processor 11 stores the generated training data in the training data DB 134 (step S16), and completes a series of processes. The processor 11 collects many first images and second images and the evaluation scores, and accumulates a plurality of information groups, generated based on the collected data in the training data DB 134 as the training data.

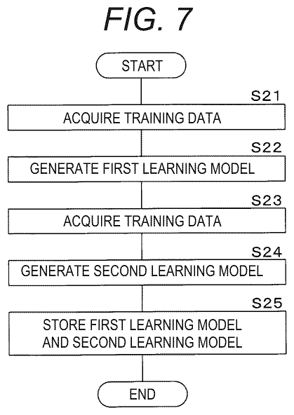

FIG. 7 is a flowchart illustrating an example of a learning model generating procedure. For example, after the process of FIG. 5 is completed in the learning phase, the following process is executed by the processor 11 according to the program 13P stored in the auxiliary storage unit 13 of the information processing apparatus 1.

The processor 11 of the information processing apparatus 1 refers to the training data DB 134 and acquires the training data in the long axis direction extracted from the information groups (step S21). The processor 11 uses the acquired training data to generate the first learning model 131 that outputs the evaluation score for the puncture line when the first image and the puncture line are input (step S22). Specifically, the processor 11 inputs the first image and the puncture line included in the training data into the first learning model 131 as the input data, and acquires the evaluation score output from the first learning model 131. The processor 11 calculates, by a predetermined loss function, an error between the output evaluation score and the evaluation score that is the ground truth value. The processor 11 adjusts a parameter such as a weight between nodes by using, for example, a back propagation method so as to optimize (minimize or maximize) the loss function. At a stage before the learning is started, it is assumed that an initial setting value is provided for definition information describing the first learning model 131. In a case where the learning is completed when the error and the number of times of learning satisfy predetermined references, the optimized parameter is obtained.

The processor 11 refers to the training data DB 134 and acquires the training data in the short axis direction extracted from the information groups (step S23). The processor 11 uses the acquired training data to generate the second learning model 132 that outputs the evaluation score for the puncture line when the second image and the puncture line are input (step S24). Specifically, the processor 11 inputs the second image and the puncture line included in the training data into the second learning model 132 as the input data, and acquires the evaluation score output from the second learning model 132. Similar to the first learning model 131 described above, the processor 11 generates the second learning model 132 by comparing the output evaluation score with the evaluation score that is the ground truth value, and optimizing the parameter such that the two evaluation scores are approximate.

When the learning is completed, the processor 11 stores, as the first learning model 131 and the second learning model 132 that are trained, the definition information on the first learning model 131 and the second learning model 132 that are trained into the auxiliary storage unit 13 (step S25), and completes the process according to the flowchart. According to the process described above, it is possible to generate the first learning model 131 that is trained to be capable of appropriately estimating the evaluation score for a puncture line in the first image. In addition, it is possible to generate the second learning model 132 that is trained to be capable of appropriately estimating the evaluation score for the puncture line in the second image.

Although an example in which the processor 11 of the information processing apparatus 1 executes a series of processes is described with reference to FIGS. 5 and 7, embodiments of this disclosure are not limited thereto. A part or all of the processes described above may be executed by the processor 31 of the image processing apparatus 3. The information processing apparatus 1 and the image processing apparatus 3 may perform a series of processes in cooperation, for example, by performing inter-process communication. The first learning model 131 and the second learning model 132 may be generated by the information processing apparatus 1 and trained by the image processing apparatus 3.

By using the first learning model 131 and the second learning model 132 generated as described above, assistance information on an optimal puncture line corresponding to a state of the blood vessel of the subject is provided in the assistance system 100. Hereinafter, a processing procedure executed by the assistance system 100 in the operation phase will be described.

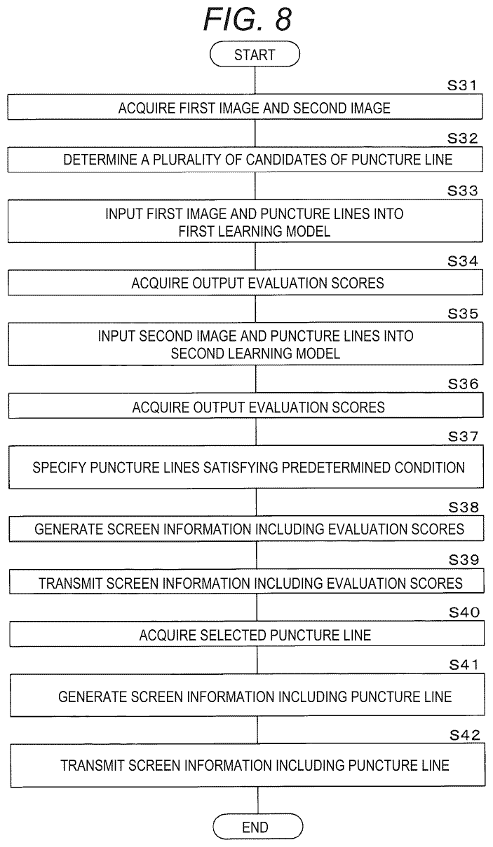

FIG. 8 is a flowchart illustrating an example of an assistance information outputting procedure. The following process is executed by the processor 11 according to the program 13P stored in the auxiliary storage unit 13 of the information processing apparatus 1. For example, each time a first image and a second image are transmitted from the image processing apparatus 3, the processor 11 performs the following process.

The processor 11 of the information processing apparatus 1 acquires the first image and the second image received from the image processing apparatus 3 (step S31). The first image and the second image are respectively ultrasonic tomographic images in the long axis direction and the short axis direction of the blood vessel, which are generated by the image processing apparatus 3 based on the echo signals received by the ultrasonic probe 2 at the same time.

The processor 11 generates a plurality of candidates of the puncture line for each of the first image and the second image (step S32). For example, the processor 11 may determine the candidates of the puncture line by appropriately selecting a predetermined number of puncture lines from the puncture line candidate table in which the start point and the angle of each puncture line are associated with each other according to the predetermined rule.

Regarding the determined puncture lines for the first image, the processor 11 inputs the first image and the puncture lines into the first learning model 131 as the input data (step S33). The processor 11 acquires the evaluation scores for the puncture lines output from the first learning model 131 (step S34).

Regarding the generated puncture lines for the second image, the processor 11 inputs the second image and the puncture lines into the second learning model 132 as the input data (step S35). The processor 11 acquires the evaluation scores for the puncture lines output from the second learning model 132 (step S36). Note that the processor 11 does not necessarily sequentially execute the estimation process by the first learning model 131 in step S33 and the estimation process by the second learning model 132 in step S35, and may execute these processes in parallel.

Based on the output results of the first learning model 131 and the second learning model 132, the processor 11 specifies one or more puncture lines satisfying a predetermined condition among all of the candidates of the puncture line for the first image and the second image (step S37). For example, the processor 11 may select a predetermined number of puncture lines from the puncture lines satisfying the condition such as the evaluation score being equal to or larger than a predetermined value, or the evaluation ranking being equal to or higher than a predetermined rank.

The processor 11 generates evaluation screen information including the evaluation scores for the specified puncture lines (step S38). The processor 11 controls the communication unit 14 to transmit the generated evaluation screen information to the image processing apparatus 3 (step S39), and the processor 31 of the image processing apparatus 3 controls the display device 4 to display an evaluation screen 154 based on the transmitted evaluation screen information.

The processor 11 acquires, for each of the first image and the second image, one puncture line selected by the operator such as a physician among the specified puncture lines (step S40). Specifically, the processor 11 determines the puncture line selected by the operator via the evaluation screen 154 displayed on the image processing apparatus 3 based on the information on the received puncture line received from the image processing apparatus 3. When the operator determines that no suitable puncture line can be obtained, and the selection of the puncture line is not made, the processor 11 may return the process to step S31 to execute the output process of the puncture lines based on a new first image and a new second image.

The processor 11 generates screen information for displaying the selected one puncture line on each of the first image and the second image in a superimposed manner (step S41). The processor 11 controls the communication unit 14 to transmit the generated screen information to the image processing apparatus 3 (step S42), which causes the processor 31 of the image processing apparatus 3 to control the display device 4 to display a screen 158 based on the transmitted screen information, and then the processor 11 completes a series of processes.

Although the example in which the processor 11 of the information processing apparatus 1 executes a series of processes is described above, embodiments of this disclosure is not limited thereto. A part or all of the process of FIG. 8 may be executed by the processor 31 of the image processing apparatus 3. The processor 31 of the image processing apparatus 3 may store the first learning model 131 and the second learning model 132 acquired from the information processing apparatus 1 into the auxiliary storage unit 33 in advance, and execute the generating process of the assistance information based on the first learning model 131 and the second learning model 132.

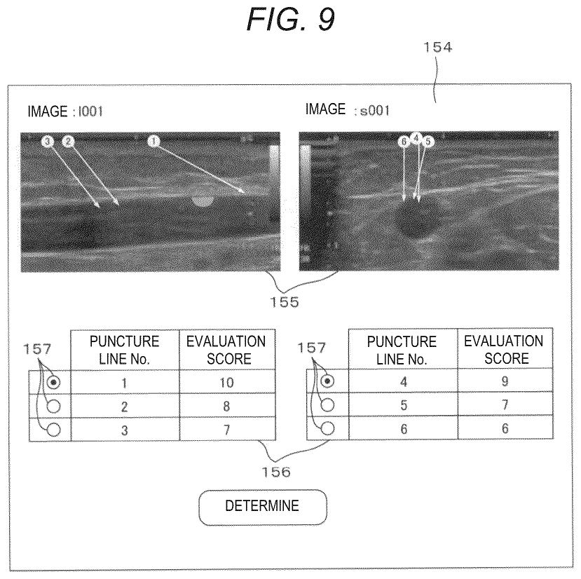

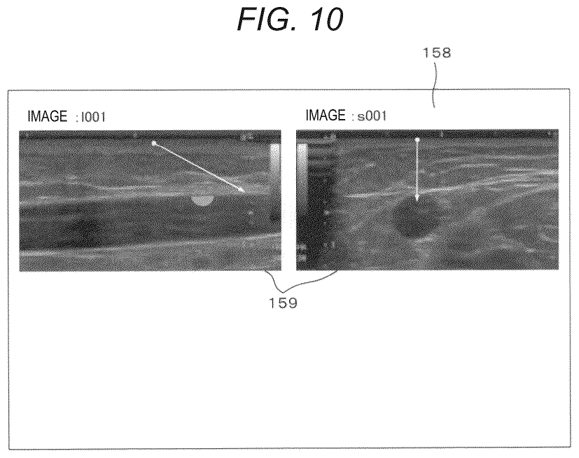

FIGS. 9 and 10 are schematic diagrams illustrating an example of a screen displayed on the display device 4. FIG. 9 is an example of the evaluation screen 154. The processor 31 of the image processing apparatus 3 controls the display device 4 to display the evaluation screen 154 as illustrated in FIG. 9 based on the evaluation screen information received from the information processing apparatus 1. The evaluation screen 154 includes a puncture line display unit area, an evaluation score display area 156, and the like. The puncture line display area 155 displays, for example, a predetermined number of puncture lines each having a high evaluation score on each of the first image and the second image in a superimposed manner. The evaluation core display area 156 displays the evaluation scores for the puncture lines, and a plurality of buttons 157 through each of which an instruction of selecting one puncture line is input by the operator using an input method such as tapping.

The processor 11 of the information processing apparatus 1 generates drawing objects based on the start point coordinates and the angles of the specified puncture lines. The generated drawing objects, each of which indicates the puncture line, are displayed on the first image and the second image. In this case, it is preferable that the processor 11 changes display modes of the puncture lines according to the evaluation scores, such as changing colors and thickness of the puncture lines according to the evaluation scores. In addition, the evaluation scores output from the learning models and the buttons 157 are displayed in association with puncture line numbers or the like assigned to the puncture lines.

The operator checks the puncture lines and the evaluation scores displayed on the evaluation screen 154, and selects one appropriate puncture line for each of the first image and the second image among the puncture lines di splayed on the evaluation screen 154. When the “determine” button is tapped in a state where the buttons 157 associated with the puncture lines selected by the operator are selected on the evaluation screen 154 of FIG. 9, the selection results of the puncture lines are input via the input device 5. The processor 31 of the image processing apparatus 3 acquires the selection results of the puncture lines, and controls the communication unit 34 to transmit the selection results of the puncture lines to the information processing apparatus 1. As described above, since the puncture lines are displayed, it is possible to perform selection corresponding to a determination, a puncture skill, and the like of the operator, and thus assistance contents are improved.

Upon receiving the selection results of the puncture lines, the processor 11 of the information processing apparatus 1 generates the screen information for displaying the screen 158 illustrated in FIG. 10, and controls the communication unit 14 to transmit the screen information to the image processing apparatus 3. The screen 158 includes a puncture line display area 159 that displays the one puncture line selected by the operator. The puncture line display area 159 processes only the one puncture line selected by the operator into, for example, a semi-transparent mask, and displays the puncture line on each of the first image and the second image in a superimposed manner. By the first image and the second image on which the puncture lines are superimposed, the inserting positions indicated by intersection points between the skin surface and the puncture lines included in the images, and the inserting directions indicated by angles of the puncture lines can be recognized.

The puncture line display area 159 may display the first image and the second image generated based on the echo signals in real time. That is, after receiving the decision on the puncture lines from the operator, the processor 11 may repeatedly execute a process of acquiring the first image and the second image generated in real time, and generating the screen information for displaying the selected puncture lines on the acquired first image and second image in a superimposed manner. After selecting the puncture line, the operator adjusts the position of the ultrasonic probe 2 and performs the puncture. The first image and the second image generated in real time include information indicating the position of the puncture device using a marker or the like. The operator performs the puncture while checking the puncture lines on the first image and the second image and the positions of the puncture device included in the first image and the second image. Specifically, the operator advances the puncture such that the positions of the puncture device on the first image and the second image are along the inserting positions and the inserting directions indicated by the puncture lines.

According to the present embodiment, the puncture performed by the operator can be suitably assisted by outputting the assistance information for guiding the inserting position and the inserting direction of the puncture device. The assistance information on the puncture lines is accurately estimated using the first learning model 131 and the second learning model 132, and is displayed in a display mode that is easily recognized by the operator. Further, by using the two-axis ultrasonic probe 2, it is possible to efficiently grasp an inserting position and an inserting direction in an orthogonal coordinate system in a two-dimensional image having two directions.

### Second Embodiment

In a second embodiment, images each showing a puncture line are output by the first learning model 131 and the second learning model 132. Hereinafter, differences from the first embodiment will be mainly described, and components common to those of the first embodiment will be denoted by the same reference numerals, and detailed description thereof will be omitted.

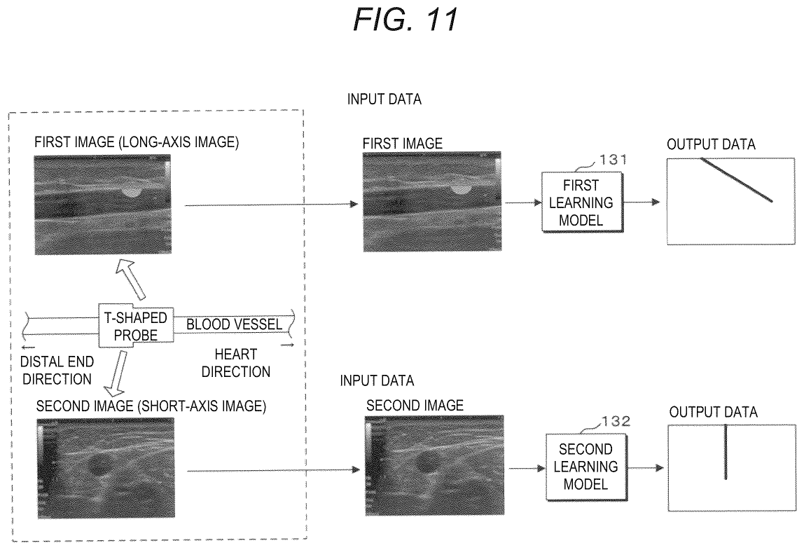

FIG. 11 is a schematic diagram illustrating configurations of the first learning model 131 and the second learning model 132 according to the second embodiment. The first learning model 131 is configured to output an image of a puncture line for the first image when the first image is input. The second learning model 132 is configured to output an image of a puncture line for the second image when the second image is input. Since the first learning model 131 and the second learning model 132 have the same configuration, the configuration of the first learning model 131 will be described.

The first learning model 131 recognizes, in units of pixels, whether each of pixels in an input image is a pixel corresponding to an object region by, for example, an image recognition technique using a semantic segmentation model. The first learning model 131 includes an input layer to which an image portion is input, an intermediate layer that extracts and restores a feature of an image, and an output layer that outputs a label image showing an object included in the image portion in units of pixels. The first learning model 131 is, for example, U-Net.

The input layer of the first learning model 131 includes a plurality of nodes that receive an input of pixel values of the pixels included in the image portion, and transmits the input pixel values to the intermediate layer. The intermediate layer includes a convolution layer (i.e., a CONV layer) and a deconvolution layer (i.e., a DECONV layer). The convolution layer is a layer that performs dimensional compression on image data. A feature of an object is extracted by the dimensional compression. The deconvolution layer performs a deconvolution process to restore the image data to an original dimension. By a restoration process in the deconvolution layer, a binarized label image indicating whether the pixels in the image are the object is generated. The output layer includes one or more nodes that output a label image. The label image is, for example, an image in which pixels corresponding to a puncture line are classified into class “1”, and pixels corresponding to other image portions are classified into class “0”.

The second learning model 132 has the same configuration as that of the first learning model 131, recognizes a puncture line included in an image portion in units of pixels, and outputs a generated label image. The label image is, for example, an image in which pixels corresponding to a puncture line are classified into class “1”, and pixels corresponding to other image portions are classified into class “0”.

The processor 11 of the information processing apparatus 1 acquires training data in which the first image generated by the image processing apparatus 3 and the puncture line for the first image are labeled for each pixel, and stores the training data into the training data DB 134. A puncture line to be a ground truth value may be acquired by, for example, receiving image data of a puncture line created by the skilled physician or the like. An untrained neural network performs machine learning using the training data, and thus it is possible to generate the first learning model 131 that is trained to be capable of appropriately estimating a puncture line for the first image. Similarly, by using training data in which the second image including a puncture line and the puncture line for the second image are labeled for each pixel, the processor 11 generates the second learning model 132 that is trained to be capable of appropriately estimating a puncture line for the second image.

In the operation phase, the processor 11 of the information processing apparatus 1 inputs the first image acquired from the image processing apparatus 3 into the first learning model 131, and acquires a label image showing a puncture line for the first image and output from the first learning model 131. Similarly, the processor 11 inputs the second image acquired from the image processing apparatus 3 into the second learning model 132, and acquires a label image showing a puncture line for the second image and output from the second learning model 132. For example, the processor 11 processes the label images output from the first learning model 131 and the second learning model 132 to semi-transparent masks, and generates image information to be superimposed on the original first image and the original second image.

According to the present embodiment, the puncture performed by the operator can be suitably assisted by generating the puncture lines which are estimated with high accuracy using the first learning model 131 and the second learning model 132.

### Third Embodiment

In a third embodiment, the first image and the second image in which an object region such as a blood vessel is extracted are acquired by the third learning model 133. Hereinafter, differences from the first embodiment will be mainly described, and components common to those of the first embodiment will be denoted by the same reference numerals, and detailed description thereof will be omitted.

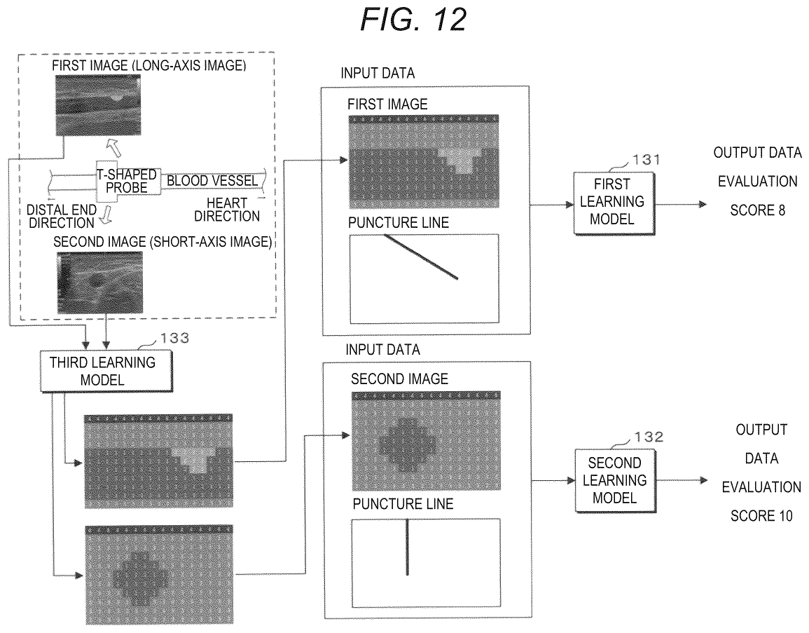

FIG. 12 is a diagram illustrating a flow of a process according to the third embodiment. The processor 11 of the information processing apparatus 1 acquires the first image and the second image from the image processing apparatus 3, and detects the object regions in the first image and the second image using the third learning model 133.

The third learning model 133 is a model that recognizes, in units of pixels, whether each of pixels in an input image is a pixel corresponding to the object region by an image recognition technique using a semantic segmentation model, and for example, is U-Net. Examples of an object to be detected by the third learning model 133 include a blood vessel, a thrombus, a subcutaneous tissue, and a skin surface. When an image including an object is input, the third learning model 133 generates a label image indicating pixels of an object region in the image. The label image is, for example, an image in which pixels corresponding to a blood vessel wall portion are classified into class “1”, pixels corresponding to a thrombus are classified into class “2”, pixels corresponding to a subcutaneous tissue are classified into class “3”, and pixels corresponding to a skin surface are classified into class “4”.

The processor 11 inputs the first image and the second image acquired from the image processing apparatus 3 into the third learning model 133, and acquires the first image and the second image in each of which the object region is detected. The processor 11 inputs the first image in which the object region is detected and a puncture line into the first learning model 131, inputs the second image in which the object region is detected and a puncture line into the second learning model 132, and outputs evaluation scores for the puncture lines.

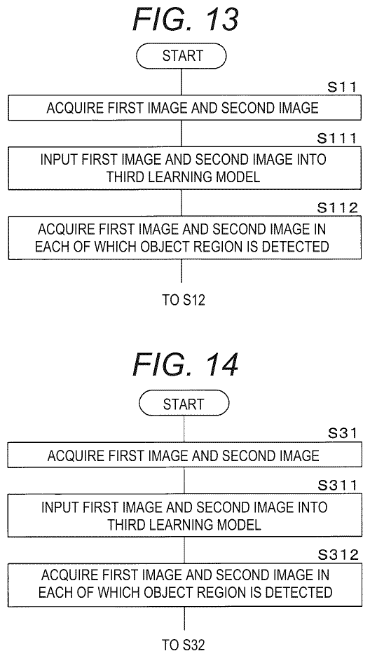

FIG. 13 is a flowchart illustrating an example of a training data generating procedure according to the third embodiment. The processor 11 of the information processing apparatus 1 acquires the first image and the second image from the image processing apparatus 3 (step S11). The first image and the second image are respectively ultrasonic tomographic images in the long axis direction and the short axis direction of the blood vessel, which are generated based on the echo signals received by the ultrasonic probe 2 at the same time.

The processor 11 inputs the acquired first image and second image into the third learning model 133 (step S111). The processor 11 acquires label images output from the third learning model 133, that is, the first image and the second image in each of which the object region is detected (step S112). The processor 11 executes the processes in S12 and subsequent steps illustrated in FIG. 5 to perform a generating process of training data including the first image and the second image in each of which the object region is detected.

In addition, the processor 11 executes the process illustrated in FIG. 6 to perform a generating process of a learning model using the generated training data. The processor 11 constructs the first learning model 131 by using training data in which the first image in which the object region is detected, the puncture line, and the evaluation score for the puncture line are labeled. The processor 11 generates the second learning model 132 by using training data in which the second image in which the object region is detected, the puncture line, and the evaluation score for the puncture line are labeled.

FIG. 14 is a flowchart illustrating an example of an assistance information outputting procedure according to the third embodiment. The processor 11 of the information processing apparatus 1 acquires the first image and the second image from the image processing apparatus 3 (step S31).

The processor 11 inputs the acquired first image and second image into the third learning model 133 (step S311). The processor 11 acquires label images output from the third learning model 133, that is, the first image and the second image in each of which the object region is detected (step S312). The processor 11 executes the processes in S32 and subsequent steps illustrated in FIG. 8 to perform an output process of the assistance information. The processor 11 inputs the first image in which the object region is detected and the puncture line into the first learning model 131, and acquires the evaluation score for the puncture line. The processor 11 inputs the second image in which the object region is detected and the puncture line into the second learning model 132, and acquires the evaluation score for the puncture line.

In the process described above, the processor 11 may perform a pre-process of extracting a region of interest from the entire image for both the first image and the second image in each of which the object region is detected. Based on the detection results of the objects, the processor 11 extracts only a particular region from each of the first image and the second image, for example, 5 cm below a skin surface including a blood vessel. The ultrasonic tomographic images generated based on the echo signals each have a wide range including a puncture target. By extracting only the region of interest necessary for generating the information on the puncture line from such an ultrasonic tomographic image, the process can be executed efficiently.

According to the present embodiment, by using the first image and the second image, in each of which the object region such as a blood vessel is detected by using the third learning model 133, as input elements of the first learning model 131 and the second learning model 132, it is possible to output information on a more preferable puncture line corresponding to a position of the blood vessel or the like.

### Fourth Embodiment

In a fourth embodiment, retraining of the first learning model 131 and the second learning model 132 is executed. Hereinafter, differences from the first embodiment will be mainly described, and components common to those of the first embodiment will be denoted by the same reference numerals, and detailed description thereof will be omitted.

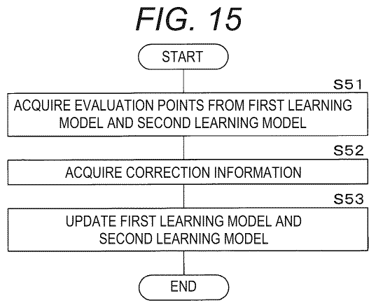

FIG. 15 is a flowchart illustrating an example of a retraining process of the learning models according to the fourth embodiment. The processor 11 of the information processing apparatus 1 acquires evaluation scores output from the first learning model 131 and the second learning model 132 (step S51). The processor 11 acquires correction information for the evaluation scores (step S52). The processor 11 may acquire the correction information based on the correction information input by the physician or the like via the image processing apparatus 3. For example, in the evaluation screen 154 illustrated in FIG. 9, the processor 31 of the image processing apparatus 3 detects a correction input for correcting the information on the evaluation scores displayed in the evaluation score display area 156, and controls the communication unit 34 to transmit the received correction information to the information processing apparatus 1. When the first learning model 131 and the second learning model 132 are models that each output an image of a puncture line, the processor 31 of the image processing apparatus 3 may acquire information on the puncture lines as the correction information.

For each of the first learning model 131 and the second learning model 132, the processor 11 performs the retraining by using the correction information for the evaluation scores to update the first learning model 131 and the second learning model 132 (step S53). Specifically, the processor 11 performs the retraining by using the first image and the puncture line input to the first learning model 131 and the correction information for the evaluation score as the training data, to update the first learning model 131. That is, the processor 11 optimizes a parameter such as a weight between nodes such that the evaluation score output from the first learning model 131 approximates the corrected evaluation score, and regenerates the first learning model 131. Similarly, the processor 11 performs the retraining by using the second image and the puncture line input to the second learning model 132 and the correction information for the evaluation score as the training data, to update the second learning model 132. Note that the processor 11 may execute the retraining process described above for only one of the first learning model 131 and the second learning model 132.

According to the present embodiment, the first learning model 131 and the second learning model 132 can be further optimized by using the assistance system 100.

The embodiments disclosed above are illustrative in all aspects, and are not restrictive. The scope of the invention is defined by the claims, and is intended to include all modifications within meanings and scopes equivalent to that of the claims. In addition, at least a part of the embodiments described above may be combined freely.

What is claimed is:

1. A medical operation assistance system for assisting in performing a medical operation on an organ, comprising:
a display device;
an ultrasonic probe; and
one or more processors configured to:
upon receipt of echo signals from the ultrasonic probe, which are generated in response to ultrasound signals transmitted by the ultrasonic probe toward the organ, generate a first image of a first cross section of the organ along a first direction and a second image of a second cross section of the organ along a second direction orthogonal to the first direction,
determine a plurality of candidates of a first puncture line indicating an inserting position and inserting direction of a puncture device in the first image,
input the first image and information indicating each of the candidates of the first puncture line to a first machine learning model and acquire first evaluation information output by the first machine learning model for each of the candidates of the first puncture line,
determine a plurality of candidates of a second puncture line indicating an inserting position and inserting direction of the puncture device in the second image,
input the second image and information indicating each of the candidates of the second puncture line to a second machine learning model and acquire second evaluation information output by the second machine learning model for each of the candidates of the second puncture line, and
control the display device to display a first screen in which each of the candidates of the first and second puncture lines is displayed in association with the corresponding image and evaluation information.

2. The medical operation assistance system according to claim 1, wherein
the first machine learning model has been trained to output evaluation information on an input puncture line in an input cross-sectional image along the first direction, and
the second machine learning model has been trained to output evaluation information on an input puncture line in an input cross-sectional image along the second direction.

3. The medical operation assistance system according to claim 1, wherein the candidates of the first and second puncture lines are superimposed over the first and second images in the first screen.

4. The medical operation assistance system according to claim 1, wherein
each of the first and second evaluation information indicates a score, and
each of the candidates of the first and second puncture lines is displayed in a different aspect depending on the score.

5. The medical operation assistance system according to claim 4, wherein the processors are configured to generate the first screen such that the candidates of the first and second puncture lines having a score higher than a threshold are displayed.

6. The medical operation assistance system according to claim 1, wherein the processors are configured to generate each of the first and second images by inputting a cross-sectional image generated from the echo signals along each of the first and second directions to a third machine learning model that has been trained to output a cross-sectional image of a predetermined part of the organ.

7. The medical operation assistance system according to claim 1, wherein the processors are configured to:
acquire correction information for correcting each of the first and second evaluation information, and
execute a retraining process for the first and second machine learning models using the acquired correction information and the first and second images.

8. The medical operation assistance system according to claim 1, wherein the processors are configured to generate the first screen including a plurality of selectable buttons each corresponding to one of the candidates of the first and second puncture lines.

9. The medical operation assistance system according to claim 8, wherein the processors are configured to control the display device to display a second screen in which the selected candidates of the first and second puncture lines are displayed over the first and second images.

10. The medical operation assistance system according to claim 1, wherein the organ is a blood vessel, and the first direction extends along the blood vessel.

11. A non-transitory computer readable medium storing a program causing a computer to execute a method of assisting in performing a medical operation on an organ, the method comprising:
acquiring a first ultrasound image of a first cross section of the organ along a first direction and a second ultrasound image of a second cross section of the organ along a second direction orthogonal to the first direction;
determining a plurality of candidates of a first puncture line indicating an inserting position and inserting direction of a puncture device in the first image;
inputting the first image and information indicating each of the candidates of the first puncture line to a first machine learning model, and acquiring first evaluation information output by the first machine learning model for each of the candidates of the first puncture line;
determining a plurality of candidates of a second puncture line indicating an inserting position and inserting direction of the puncture device in the second image;
inputting the second image and information indicating each of the candidates of the second puncture line to a second machine learning model, and acquiring second evaluation information output by the second machine learning model for each of the candidates of the second puncture line; and
displaying a first screen in which each of the candidates of the first and second puncture lines is displayed in association with the corresponding image and evaluation information.

12. The non-transitory computer readable medium according to claim 11, wherein the method further comprising:
training the first machine learning model to output evaluation information on an input puncture line in an input cross-sectional image along the first direction; and
training the second machine learning model to output evaluation information on an input puncture line in an input cross-sectional image along the second direction.

13. The non-transitory computer readable medium according to claim 11, wherein the candidates of the first and second puncture lines are superimposed over the first and second images in the first screen.

14. The non-transitory computer readable medium according to claim 11, wherein
each of the first and second evaluation information indicates a score, and
each of the candidates of the first and second puncture lines is displayed in a different aspect depending on the score.

15. The non-transitory computer readable medium according to claim 14, wherein the method further comprises:
generating the first screen such that the candidates of the first and second puncture lines having a score higher than a threshold are displayed.

16. The non-transitory computer readable medium according to claim 11, wherein
acquiring the first and second images includes inputting a cross-sectional image generated from the echo signals along each of the first and second directions to a third machine learning model that has been trained to output a cross-sectional image of a predetermined part of the organ.

17. The non-transitory computer readable medium according to claim 11, wherein the method further includes:
acquiring correction information for correcting each of the first and second evaluation information; and
executing a retraining process for the first and second machine learning models using the acquired correction information and the first and second images.

18. The non-transitory computer readable medium according to claim 11, wherein the method further comprises:
generating the first screen including a plurality of selectable buttons each corresponding to one of the candidates of the first and second puncture lines.

19. The non-transitory computer readable medium according to claim 18, wherein the method further comprises:
displaying a second screen in which the selected candidates of the first and second puncture lines are displayed over the first and second images.

20. A medical operation assistance system for assisting in performing a medical operation on an organ, comprising:
a display device;
an ultrasonic probe; and
one or more processors configured to:
upon receipt of echo signals from the ultrasonic probe, which are generated in response to ultrasound signals transmitted by the ultrasonic probe toward the organ, generate a first image of a first cross section of the organ along a first direction and a second image of a second cross section of the organ along a second direction orthogonal to the first direction,
input the first image to a first machine learning model, and acquire an image of a first puncture line output by the first machine learning model, the first puncture line indicating an inserting position and inserting direction of a puncture device in the first image,
input the second image to a second machine learning model, and acquire an image of a second puncture line output by the second machine learning model, the second puncture line indicating an inserting position and inserting direction of the puncture device in the second image, and
control the display device to display a first screen in which the first and second puncture lines are displayed over the first and second image.
